(19) United States
(12) Patent Application Publication
(10) Pub. No.: US 2017/0239090 A1
(43) Pub. Date: Aug. 24, 2017

(54) PROTECTIVE GOGGLE

(71) Applicant: MYUNG IL HAN, Cheongju-si (KR)

(72) Inventor: MYUNG IL HAN, Cheongju-si (KR)

(21) Appl. No.: 15/337,104

(22) Filed: Oct. 28, 2016

(30) Foreign Application Priority Data
Feb. 24, 2016 (KR) 20-2016-0001003

(51) Int. Cl. A61F 9/02

(52) U.S. Cl. CPC A61F 9/028

(57) ABSTRACT

There is provided a protective goggle comprising: a lens frame to support at least one lens; and an anti-fogging and dust-removal assembly disposed in or on the frame, wherein the anti-fogging and dust-removal assembly is configured to suction an air on an inner face of the lens and discharge the suctioned air onto an outer face of the lens. The assembly provides concurrently both functions of anti-fogging and dust-removal on the lens of the goggle.

### CROSS-REFERENCE TO RELATED APPLICATION

This application claims the benefit of Korea patent application No. 10-2016-0001003, filed on Feb. 24, 2016, the entire content of which is incorporated herein by reference for all purposes as if fully set forth herein.

### BACKGROUND

Field of the Present Disclosure

The present disclosure relates to a protective goggle, and, more particularly, to a protective goggle having an anti-fogging and dust removal assembly configured to remove the fog and dust on a lens.

Discussion of Related Art

Regarding a prior-art protective goggle, Korean patent application number 2013-152003 discloses anti-glare electronic goggles that protect eyes from harmful glare, have excellent wearing sensation by arranging an adjustment part and a battery on the lens portion of a front surface and on the rear surface of a bow to balance weight arranged ergonomically at front and rear parts, and are convenient to wear by forming inner and outer soft housings by double-injection so that the goggles can adhere closely to the face of a wearer for goggle-wearer's convenience.

Korean patent application number 2014-7024562 discloses an eye-shield condensation preventing system comprising an eye shield adapted for protecting a user's eyes and adapted for defining at least a partially enclosed space between the user's eyes and the eye shield, a power source, a pulse-width modulator preferably comprised of a microcomputer, switching means, preferably comprised of a MOSFET device, that is responsive to the pulse-width modulator, a preferably indium-tin-oxide (ITO) heating element on the eye shield, and a circuit interconnecting the power source, the pulse-width modulator, the switching means and the heating element for controlling heating of the eye shield. In one embodiment the eye shield is divided into a plurality of heating regions. A current adjustment means, sensors, and optionally user-selectable profiles are employed to allow heating adjustment or automated anti-fog capability and variable condition adaptability of the invention.

The above prior art protective goggle may not provide concurrently both functions of anti-fogging and dust-removal on the lens of the goggle.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure is to provide a protective goggle to provide concurrently both functions of anti-fogging and dust-removal on the lens of the goggle.

In one aspect of the present disclosure, there is provided a protective goggle comprising: a lens frame to support at least one lens; and an anti-fogging and dust-removal assembly disposed in or on the frame, wherein the anti-fogging and dust-removal assembly is configured to suction an air on an inner face of the lens and discharge the suctioned air onto an outer face of the lens, wherein the anti-fogging and dust-removal assembly includes: an air suction duct disposed in the lens frame, wherein the air suction duct has a plurality of first and second air holes at top and bottom portions thereof defined therein respectively; an air suction blade disposed in the air suction duct; a motor configured to rotate the air suction blade; a power supply disposed in or on the lens frame to supply a power to the motor; and an air discharge duct disposed in and out of the lens frame to air-communicate with the air suction duct.

In one implementation, the air discharge duct partially extends horizontally over the air suction duct to be spaced from the air suction, and partially vertically extends outside of the lens frame.

In one implementation, the air discharge duct has a protrusion end to be oriented toward an outer face of the lens.

In one implementation, the lens frame has an upper portion having a larger thickness than that of a lower portion thereof, wherein the anti-fogging and dust-removal assembly is disposed in the upper portion.

In one implementation, a door is disposed in the upper portion of the lens frame for replacing the power supply.

In one implementation, the anti-fogging and dust-removal assembly further include a charging USB terminal in or on the lens frame to be electrically coupled to the power supply.

In one implementation, the air discharge duct has a guide plate therein to guide the suctioned air therein.

In one implementation, the anti-fogging and dust-removal assembly has a porous plate attached to the protrusion end of the air discharge duct.

In one implementation, the anti-fogging and dust-removal assembly has a length-adjustable nozzle attached to the protrusion end of the air discharge duct.

In one implementation, the length-adjustable nozzle operates in a telescopic manner.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 illustrates a front view of a protective goggle in accordance with a first embodiment of the present disclosure.

FIG. 2 illustrates a cross-sectional view of a protective goggle taken in a line A-A in FIG. 2.

FIG. 3 illustrates an enlarged view of a “B” portion in FIG. 2.

FIG. 4 illustrates an enlarged view of a key portion of a protective goggle in accordance with a second embodiment of the present disclosure.

FIG. 5 illustrates an enlarged view of a key portion of a protective goggle in accordance with a third embodiment of the present disclosure.

FIG. 6 illustrates an enlarged view of a key portion of a protective goggle in accordance with a fourth embodiment of the present disclosure.

FIG. 7 illustrates an enlarged view of a key portion of a protective goggle in accordance with a fifth embodiment of the present disclosure.

FIG. 8 illustrates a top view of a porous plate in FIG. 7.

For simplicity and clarity of illustration, elements in the figures are not necessarily drawn to scale. The same reference numbers in different figures denote the same or similar elements, and as such perform similar functionality. Also, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

### DETAILED DESCRIPTIONS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

Example embodiments will be described in more detail with reference to the accompanying drawings. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, s, operations, elements, and/or components, but do not preclude the presence or addition of one or more other features, integers, s, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

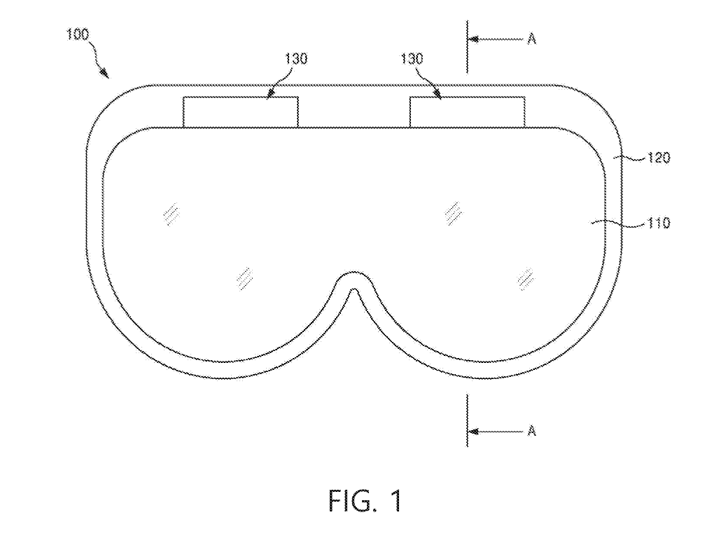

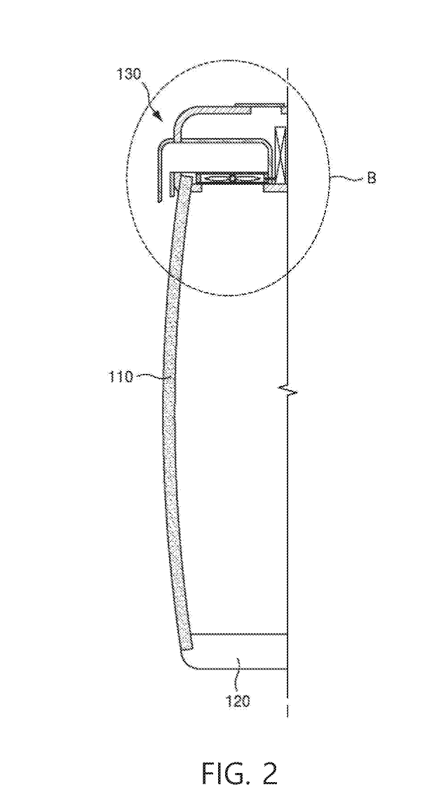

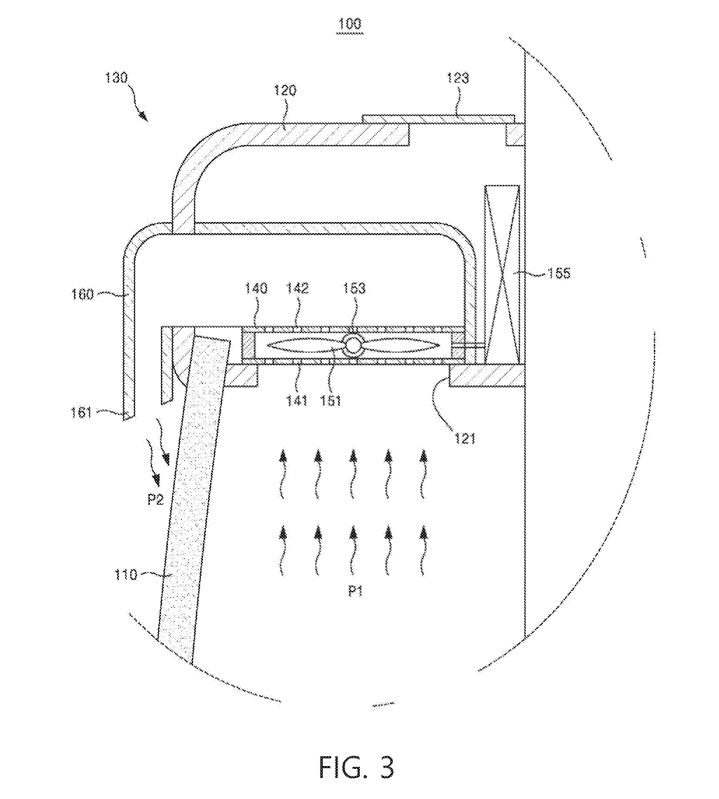

FIG. 1 illustrates a front view of a protective goggle in accordance with a first embodiment of the present disclosure. FIG. 2 illustrates a cross-sectional view of a protective goggle taken in a line A-A in FIG. 2. FIG. 3 illustrates an enlarged view of a “B” portion in FIG. 2.

Referring to FIG. 1 to FIG. 3, the protective goggle 100 in accordance with a first embodiment of the present disclosure may include a lens 110, a lens frame 120 and an anti-fogging and dust-removal assembly 130 configured to remove the fog and dust on the lens 110.

The lens frame 120 may be configured to support the lens 110. The lens frame 120 may be made of a plastic.

The lens frame 120 may surround the lens 110. In this embodiment, the lens frame 120 may have an upper portion having a larger thickness than that of a lower portion since the anti-fogging and dust-removal assembly 130 may be disposed in the upper portion.

The anti-fogging and dust-removal assembly 130 may be configured to suction an air between the lens 110 and the user' eye portion as indicated by an arrow P1 in FIG. 3 and discharge the suctioned air outside of the goggle 100 as indicated by an arrow P2 in FIG. 3. In this way, the anti-fogging and dust-removal assembly 130 may function to suppress a fog and remove a dust on an inner face of the lens 110.

During a long time wearing of the protective goggle 100, a temperature difference between an ambient and the space between the lens and the user's eye portion may lead to a fogging on the inner face of the lens 110. This fogging may block the sight of the user and thus lead to a risk of the damage. Further, a precision for a work achieved by the user may be poor. Thus, the anti-fogging and dust-removal assembly 130 may suppress a fog and remove a dust on an inner face of the lens 110, to prevent the risk of the damage and to allow the user to achieve the precision for the work.

Further, the anti-fogging and dust-removal assembly 130 may discharge the suctioned air outside of the protective goggle 100 to remove the dust deposited on the inner face of the lens 110.

In terms of a configuration, the anti-fogging and dust-removal assembly 130 may include an air suction duct 140, an air suction blade 151, a motor 153, a battery 155, and an air discharge duct 160. In this embodiment, a pair of the anti-fogging and dust-removal assemblies 130 is disposed. However, the present disclosure is not limited thereto. A single anti-fogging and dust-removal assembly 130 may extend horizontally over the entire upper portion of the lens frame 120.

The air suction duct 140 may be disposed in an opening 121 in the lens frame 120. The air suction duct 140 may have a plurality of first and second air holes 141,142. When the air suction blade 151 rotates, an air in the space between the lens 110 and the user's eye portion may be suctioned through the first and second air holes 141,142 in the air suction duct 140 to the air discharge duct 160.

The air suction blade 151 may be disposed in the air suction duct 140. The air suction blade 151 may be structured such that, when the air suction blade 151 rotates, the air in the space between the lens 110 and the user's eye portion may be suctioned through the first and second air holes 141,142 in the air suction duct 140.

The motor 153 may drive the air suction blade 151. The battery 155 may be disposed in the lens frame 120 to supply the power to the motor 153. Although not shown, an electrical connection between the motor and the battery may be formed.

The battery 155 may be replaced. To this end, in order to replace the battery 155, the lens frame 120 may have a door 123 defined at the top thereof. The door 123 may be a slide or hinge type.

The air discharge duct 160 may be formed to horizontally extend over the air suction duct 140 to be spaced from the suction duct 140 and to vertically extend downwardly. The air discharge duct 160 may have a protrusion end 161 to discharge the suctioned air toward an outer face of the lens 110. The air discharge duct has the protrusion end to be oriented toward an outer face of the lens 110.

The operation of the protective goggle 100 may be as follows: the motor 153 may rotate to rotate the air suction blade 151 to suction the air in the space between the lens 110 and the user' eye portion as indicated by an arrow P1 in FIG. 3. Then, the suctioned air may pass through the first and second air holes 141,142 in the air suction duct 140 to the air discharge duct 160. Then, the air may be discharged outside of the goggle 100 as indicated by an arrow P2 in FIG. 3. In this way, the anti-fogging and dust-removal assembly 130 may function to suppress a fog and remove a dust on an inner face of the lens 110.

In this connection, when the air is discharged through the air discharge duct 160 outside of the goggle 100, the air may be guided via the protrusion end 161 of the air discharge duct 160 toward the outer face of the lens 110. In this way, the dust deposited on the outer face of the lens 110 may be removed. Thus, the dusts on the inner face and outer face of the lens 110 may be removed at the same time.

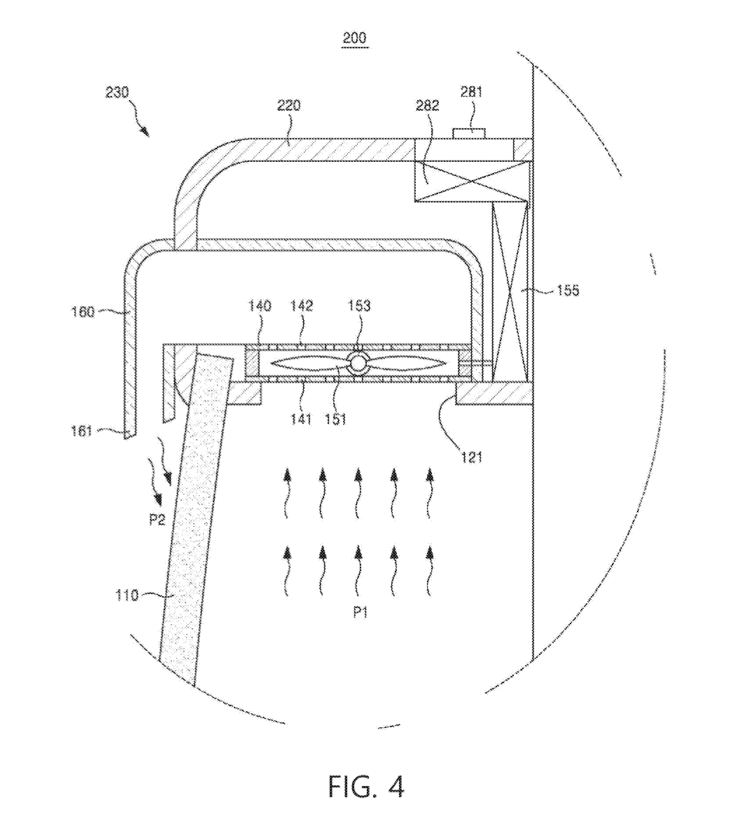

FIG. 4 illustrates an enlarged view of a key portion of a protective goggle in accordance with a second embodiment of the present disclosure. Referring to FIG. 4, the protective goggle 200 in accordance with a second embodiment of the present disclosure may include a lens 110, a lens frame 220 and an anti-fogging and dust-removal assembly 230 configured to remove the fog and dust on the lens 110.

The anti-fogging and dust-removal assembly 230 may be configured to suction an air between the lens 110 and the user' eye portion as indicated by an arrow P1 in FIG. 4 and discharge the suctioned air outside of the goggle 200 as indicated by an arrow P2 in FIG. 4. In this way, the anti-fogging and dust-removal assembly 230 may function to suppress a fog and remove a dust on an inner face of the lens 110.

During a long time wearing of the protective goggle 200, a temperature difference between an ambient and the space between the lens and the user's eye portion may lead to a fogging on the inner face of the lens 110. This fogging may block the sight of the user and thus lead to a risk of the damage. Further, a precision for a work achieved by the user may be poor. Thus, the anti-fogging and dust-removal assembly 230 may suppress a fog and remove a dust on an inner face of the lens 110, to prevent the risk of the damage and to allow the user to achieve the precision for the work.

In terms of a configuration, the anti-fogging and dust-removal assembly 230 may include an air suction duct 140, an air suction blade 151, a motor 153, a battery 155, and an air discharge duct 160.

The air suction blade 151 may be disposed in the air suction duct 140. The air suction blade 151 may be structured such that, when the air suction blade 151 rotates, the air in the space between the lens 110 and the user's eye portion may be suctioned through the first and second air holes 141,142 in the air suction duct 140. The motor 153 may drive the air suction blade 151. The battery 155 may be disposed in the lens frame 120 to supply the power to the motor 153. Although not shown, an electrical connection between the motor and the battery may be formed. The air discharge duct 160 may be formed to horizontally extend over the air suction duct 140 to be spaced from the suction duct 140 and to vertically extend downwardly, The air discharge duct 160 may have a protrusion end 161 to discharge the suctioned air toward an outer face of the lens 110.

In this second embodiment, the anti-fogging and dust-removal assembly 230 may further include a charging USB terminal 281 electrically coupled to the battery 155. The charging USB terminal 281 may be disposed in the lens frame 220. The charging USB terminal 281 may be coupled to the battery 155 via a PCB 282.

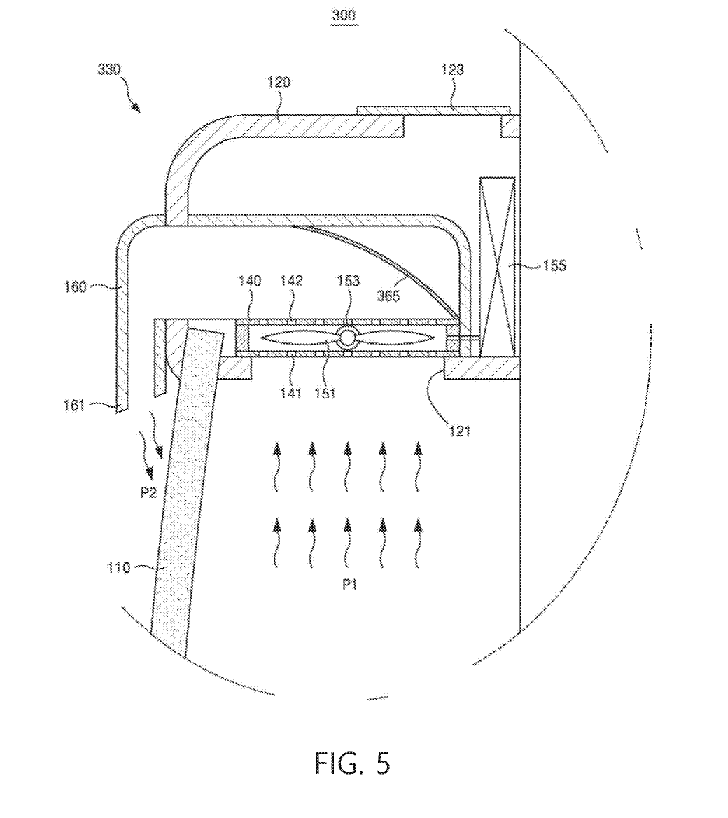

FIG. 5 illustrates an enlarged view of a key portion of a protective goggle in accordance with a third embodiment of the present disclosure. Referring to FIG. 5, the protective goggle 300 in accordance with a third embodiment of the present disclosure may include a lens 110, a lens frame 120 and an anti-fogging and dust-removal assembly 330 configured to remove the fog and dust on the lens 110.

The anti-fogging and dust-removal assembly 330 may be configured to suction an air between the lens 110 and the user' eye portion as indicated by an arrow P1 in FIG. 5 and discharge the suctioned air outside of the goggle 300 as indicated by an arrow P2 in FIG. 5. In this way, the anti-fogging and dust-removal assembly 330 may function to suppress a fog and remove a dust on an inner face of the lens 110.

During a long time wearing of the protective goggle 300, a temperature difference between an ambient and the space between the lens and the user's eye portion may lead to a fogging on the inner face of the lens 110. This fogging may block the sight of the user and thus lead to a risk of the damage. Further, a precision for a work achieved by the user may be poor. Thus, the anti-fogging and dust-removal assembly 330 may suppress a fog and remove a dust on an inner face of the lens 110, to prevent the risk of the damage and to allow the user to achieve the precision for the work.

In terms of a configuration, the anti-fogging and dust-removal assembly 330 may include an air suction duct 140, an air suction blade 151, a motor 153, a battery 155, and an air discharge duct 160.

The air suction blade 151 may be disposed in the air suction duct 140. The air suction blade 151 may be structured such that, when the air suction blade 151 rotates, the air in the space between the lens 110 and the user's eye portion may be suctioned through the first and second air holes 141,142 in the air suction duct 140. The motor 153 may drive the air suction blade 151. The battery 155 may be disposed in the lens frame 120 to supply the power to the motor 153. Although not shown, an electrical connection between the motor and the battery may be formed. The air discharge duct 160 may be formed to horizontally extend over the air suction duct 140 to be spaced from the suction duct 140 and to vertically extend downwardly. The air discharge duct 160 may have a protrusion end 161 to discharge the suctioned air toward an outer face of the lens 110.

In this third embodiment, in the air discharge duct 160, a guide plate 365 may be formed to guide the air therein. The guide plate 365 may guide the suctioned air from the inner face of the lens 110 without an eddy air flow in the air discharge duct 160.

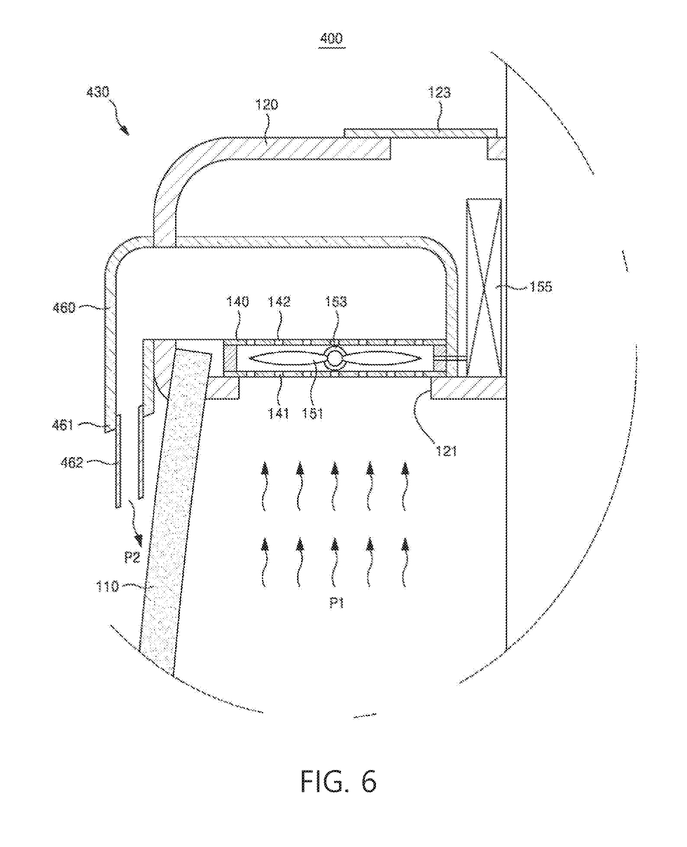

FIG. 6 illustrates an enlarged view of a key portion of a protective goggle in accordance with a fourth embodiment of the present disclosure. Referring to FIG. 5, the protective goggle 400 in accordance with a fourth embodiment of the present disclosure may include a lens 110, a lens frame 120 and an anti-fogging and dust-removal assembly 430 configured to remove the fog and dust on the lens 110.

The anti-fogging and dust-removal assembly 430 may be configured to suction an air between the lens 110 and the user' eye portion as indicated by an arrow P1 in FIG. 6 and discharge the suctioned air outside of the goggle 300 as indicated by an arrow P2 in FIG. 6. In this way, the anti-fogging and dust-removal assembly 430 may function to suppress a fog and remove a dust on an inner face of the lens 110.

During a long time wearing of the protective goggle 400, a temperature difference between an ambient and the space between the lens and the user's eye portion may lead to a fogging on the inner face of the lens 110. This fogging may block the sight of the user and thus lead to a risk of the damage. Further, a precision for a work achieved by the user may be poor. Thus, the anti-fogging and dust-removal assembly 430 may suppress a fog and remove a dust on an inner face of the lens 110, to prevent the risk of the damage and to allow the user to achieve the precision for the work.

In terms of a configuration, the anti-fogging and dust-removal assembly 430 may include an air suction duct 140, an air suction blade 151, a motor 153, a battery 155, and an air discharge duct 460.

The air suction blade 151 may be disposed in the air suction duct 140. The air suction blade 151 may be structured such that, when the air suction blade 151 rotates, the air in the space between the lens 110 and the user's eye portion may be suctioned through the first and second air holes 141,142 in the air suction duct 140. The motor 153 may drive the air suction blade 151. The battery 155 may be disposed in the lens frame 120 to supply the power to the motor 153. Although not shown, an electrical connection between the motor and the battery may be formed. The air discharge duct 460 may be formed to horizontally extend over the air suction duct 140 to be spaced from the suction duct 140 and to vertically extend downwardly. The air discharge duct 460 may have a protrusion end 461 to discharge the suctioned air toward an outer face of the lens 110.

In this fourth embodiment, the protrusion end 461 of the air discharge duct 460 may have a length-adjustable nozzle 462. The length-adjustable nozzle 462 may operate in a telescopic manner. The length-adjustable nozzle 462 may operate manually or using a power from the motor. The length-adjustable nozzle 462 may taper with a distal end with the smallest diameter. This may increase the air pressure on the outer face of the lens 110 to improve the dust removal efficiency.

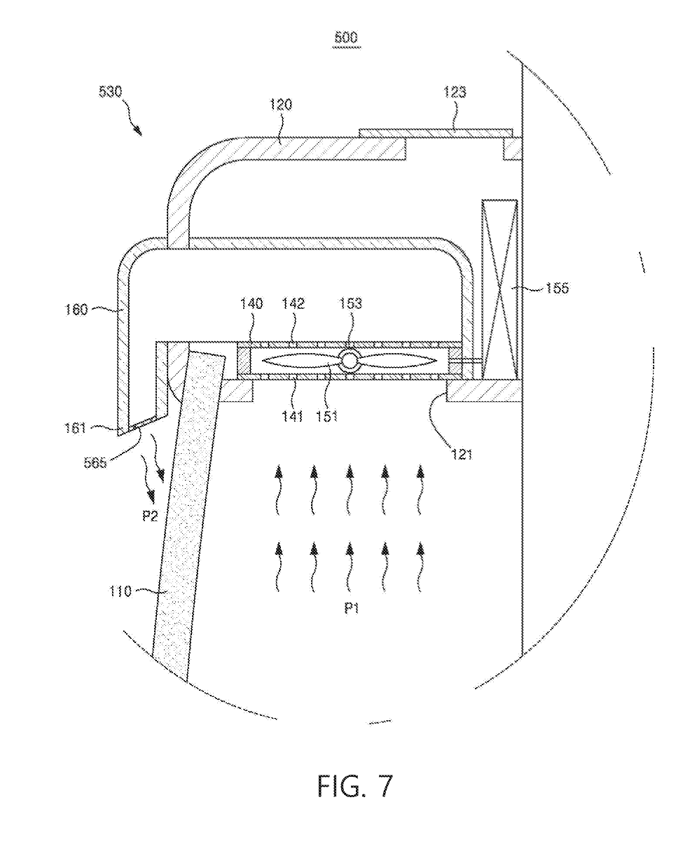

FIG. 7 illustrates an enlarged view of a key portion of a protective goggle in accordance with a fifth embodiment of the present disclosure. Referring to FIG. 7, the protective goggle 500 in accordance with a fifth embodiment of the present disclosure may include a lens 110, a lens frame 120 and an anti-fogging and dust-removal assembly 530 configured to remove the fog and dust on the lens 110.

The anti-fogging and dust-removal assembly 530 may be configured to suction an air between the lens 110 and the user' eye portion as indicated by an arrow P1 in FIG. 7 and discharge the suctioned air outside of the goggle 300 as indicated by an arrow P2 in FIG. 7. In this way, the anti-fogging and dust-removal assembly 530 may function to suppress a fog and remove a dust on an inner face of the lens 110.

During a long time wearing of the protective goggle 500, a temperature difference between an ambient and the space between the lens and the user's eye portion may lead to a fogging on the inner face of the lens 110. This fogging may block the sight of the user and thus lead to a risk of the damage. Further, a precision for a work achieved by the user may be poor. Thus, the anti-fogging and dust-removal assembly 530 may suppress a fog and remove a dust on an inner face of the lens 110, to prevent the risk of the damage and to allow the user to achieve the precision for the work.

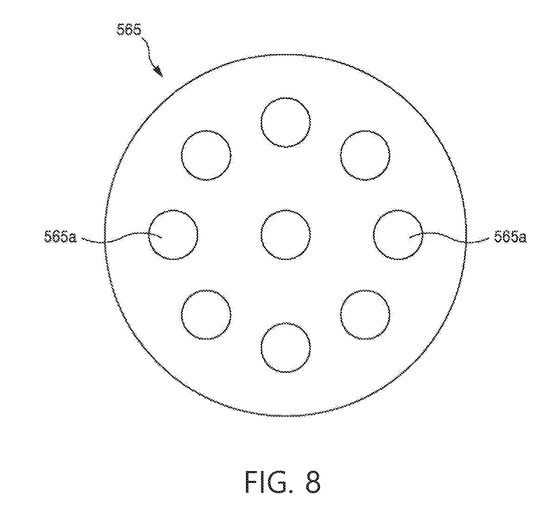

In this fifth embodiment, the anti-fogging and dust-removal assembly 530 may have a porous plate 565 attached to the protrusion end 161 of the air discharge duct 160. FIG. 8 illustrates a porous plate. The porous plate 565 may have multiple air holes 565a defined therein. This may increase the air pressure on the outer face of the lens 110 to improve the dust removal efficiency.

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

What is claimed is:

1. A protective goggle comprising:
A lens frame to support at least one lens; and
an anti-fogging and dust-removal assembly disposed in or on the frame, wherein the anti-fogging and dust-removal assembly is configured to suction an air on an inner face of the lens and discharge the suctioned air onto an outer face of the lens,
wherein the anti-fogging and dust-removal assembly includes:
an air suction duct disposed in the lens frame, wherein the air suction duct has a plurality of first and second air holes at top and bottom portions thereof defined therein respectively;
an air suction blade disposed in the air suction duct;
a motor configured to rotate the air suction blade;
a power supply disposed in or on the lens frame to supply a power to the motor; and
an air discharge duct disposed in and out of the lens frame to air-communicate with the air suction duct.

2. The goggle of claim 1, wherein the air discharge duct partially extends horizontally over the air suction duct to be spaced from the air suction, and partially vertically extends outside of the lens frame.

3. The goggle of claim 1, wherein the air discharge duct has a protrusion end to be oriented toward an outer face of the lens.

4. The goggle of claim 1, wherein the lens frame has an upper portion having a larger thickness than that of a lower portion thereof, wherein the anti-fogging and dust-removal assembly is disposed in the upper portion.

5. The goggle of claim 4, wherein a door is disposed in the upper portion of the lens frame for replacing the power supply.

6. The goggle of claim 1, wherein the anti-fogging and dust-removal assembly further include a charging USB terminal in or on the lens frame to be electrically coupled to the power supply.

7. The goggle of claim 1, wherein the air discharge duct has a guide plate therein to guide the suctioned air therein.

8. The goggle of claim 3, wherein the anti-fogging and dust-removal assembly has a porous plate attached to the protrusion end of the air discharge duct.

9. The goggle of claim 3, wherein the anti-fogging and dust-removal assembly has a length-adjustable nozzle attached to the protrusion end of the air discharge duct.

10. The goggle of claim 9, wherein the length-adjustable nozzle operates in a telescopic manner.
